SEQUENCE LISTING (4 sequences)

SEQ ID NO: 1 - protein, 371 aa, Virus of classical swine fever
  E2 protein of classical swine fever virus
RLACKEDYRYAISSTNEIGLLGAEGLTTTWKEYSHGLQLDDGTVKAVCTAGSFKVTALNVVSRRYLASLHKRALPTSVTFELLFDGTNPAIEEMDDDFGFGLCPFDTSPVIKGKYNTTLLNGSAFYLVCPIGWTGVVECTAVSPTTLRTEVVKTFRRDKPFPHRVDCVTTIVEKEDLFHCKLGGNWTCVKGDPVTYKGGQVKQCRWCGFEFKEPYGLPHYPIGKCILTNETGYRVVDSTDCNRDGVVISTEGEHECLIGNTTVKVHALDERLGPMPCRPKEIVSSEGPVRKTSCTFNYTKTLRNKYYEPRDSYFQQYMLKGEYQYWFNLDVTDHHTDYFAEFVVLVVVALLGGRYVLWLIVTYIILTEQLA

SEQ ID NO: 2 - DNA, 1120 bp, Virus of classical swine fever
  Codon-optimized E2 gene of the classical swine fever virus
cgcctcgcctgcaaggaggactaccggtacgcgatcagcagcaccaacgagatcggcctgctcggcgccgaggggctgaccacgacctggaaggagtactcccacggcctccagctggacgacggcacggtcaaggcggtgtgcaccgccgggagcttcaaggtcaccgcgctcaacgtggtctcgcgccggtacctggccagcctccacaagcgcgccctgccgacgagcgtgaccttcgagctgctgttcgacggcaccaaccccgccatcgaggagatggacgacgacttcggcttcgggctctgcccgttcgacacgtcccccgtcatcaagggcaagtacaacaccaccctgctcaacgggtcggcgttctacctggtgtgcccgatcggctggacgggcgtcgtggagtgcaccgccgtcagccccaccacgctccggaccgaggtggtcaagacgttccgccgggacaagccgttcccccaccgcgtggactgcgtcaccacgatcgtggagaaggaggacctgttccactgcaagctcgggggcaactggacctgcgtcaagggcgacccggtgacgtacaaggggggccaggtcaagcagtgccggtggtgcggcttcgagttcaaggagccctacgggctgccgcactaccccatcggcaagtgcatcctcaccaacgagaccggctaccgcgtggtcgactccacggactgcaaccgggacggggtggtcatctcgaccgagggcgagcacgagtgcctgatcggcaacaccacggtgaaggtccacgcgctcgacgagcgcctggggccgatgccctgccggccgaaggagatcgtgtcctccgagggccccgtccgcaagacctcgtgcaccttcaactacacgaagaccctccggaacaagtactacgagccgcgcgacagctacttccagcagtacatgctgaagggggagtaccagtactggttcaacctcgacgtgaccgaccaccacacggactacttcgccgagttcgtcgtgctggtcgtggtcgcgctcctgggcggccggtacgtgctctggctgatcgtcacctacatcatcctcaccgagcagctggcctgagctc

SEQ ID NO: 3 - DNA, 434 bp, Hordeum vulgare
  Hordein promoter including sequence for the signal peptide
cttcgagtgcccgccgatttgccagcaatggctaacagacacatattctgccaaaaccccagaacaataatcacttctcgtagatgaagagaacagaccaagatacaaacgtccacgcttcagcaaacagtaccccagaactaggattaagccgattacgcggctttagcagaccgtccaaaaaaactgttttgcaaagctccaattcctccttgcttatccaatttcttttgtgttggcaaactgcacttgtccaaccgattttgttcttcccgtgtttcttcttaggctaactaacacagccgtgcacatagccatggtccggaatcttcacctcgtccctataaaagcccagccaatctccacaatctcatcatcaccgagaacaccgagaaccacaaaactagagatcaattcattgacagtccaccgag

SEQ ID NO: 4 - DNA, 777 bp, Hordeum vulgare
  Amylase promoter including sequence for the signal peptide
ttcacacattcaaaaaaagcaaatttcaaaatttgtactcgaatttaaaaaaagatcattgatttaaaaaatttcgctaagtaaaaaactgtccacgtatttcaaaaaaagataagtgggcatcaatcggcaaaatttgatatgtcagggtattgagccatattaatttggttgtggcacaatatttttgtcccccgttgcaacgcacggttatttttgttagtgtgttcttatggatatatacgcccataaagtttaagcaatacaacaactatttcaccaatccctctattctctttcttcacagcagatagaaggttttgtaattgtaaccacagcacactattcgatgaaaaatgcatcgaatgttctgtcctcagaaaaacagaggttgaggataactgacggtcgtattgatccggtgccttcttatggaaggccaaggctgcctccatctacatcacttgggacattgaatcgccttttgagctcaccgtaccggccgataacaaactccggctgacatatccactggcccaaaggagcattgaagccgagcacacgagaaagtgatttgcaagttgcacaccggcagcaattccggcatgctgcagcacactataaatacctggccagacacacaagttgaatgcatcagttctccatcgtactcttcgagagcacagcaagagagagctgaagaacatggcgaacaaacatttgtccctctccctcttcctcgtcctccttggcctgtcggccagcttggcctccggg

(19) United States
(12) Patent Application Publication
(10) Pub. No.: US 2003/0077640 A1
(43) Pub. Date: Apr. 24, 2003

(54) Process for producing a marker vaccine against a mammalian virus

(75) Inventors: Rainer Stahl, Bonn (DE); Birgit Nelsen-Salz, Koln (DE); Norbert Wolf, Andernach (DE)

Correspondence Address: LEYDIG VOIT & MAYER, LTD, TWO PRUDENTIAL PLAZA, SUITE 4900, 180 NORTH STETSON AVENUE, CHICAGO, IL, 60601-6780, US

(73) Assignee: Maltagen Forschung GMBH, Andernach (DE)

(21) Appl. No.: 10/246,243

(22) Filed: Sep. 18, 2002

(30) Foreign Application Priority Data
Sep. 18, 2001 (DE) 10145969.6

(51) Int. Cl. A01H 5/00; A61K 39/12; C12N 15/82; C12Q 1/68; A01H 1/00; C12N 15/87

(52) U.S. Cl. 435/6; 800/288; 800/320.1; 424/186.1

(57) ABSTRACT

The present invention relates to a process for producing a marker vaccine against a mammalian virus and to transgenic cereal plants or parts thereof which produce the components of the vaccine. In particular, a vaccine for combating classical swine fever is provided.

### FIELD OF THE INVENTION

The present invention relates to a process for producing a marker vaccine against a mammalian virus as well as transgenic cereal plants or parts thereof, which produce components of the vaccine.

### BACKGROUND OF THE INVENTION

Viral infections of cattle, pigs, domestic fowl and other animals used for producing meat regularly cause very high financial losses in agriculture, since no or only insufficient methods of therapeutic treatment, or no or only insufficiently efficient vaccines are available against a large number of viral infectious diseases of these animals. Alone in the years between 1995 and 2000, more than one million pigs had to be culled so as to eliminate classical swine fever (CSFV). In the European Union, the financial losses caused by CSFV in the period between 1993 and 1997 are estimated at 5 billion ECU.

However, CSFV occurs not only among domestic pigs but also among our indigenous wild boars. This species has again been attacked to an increasing extent by the epidemic since the early nineties. Wild boars infected with the CSFV virus were in the last view years also an important source of infection for our domestic pig population. 55% of the outbreaks of CSFV among domestic pigs in the period between 1993 and 1999 were considered to have originated from direct and indirect contacts with infected wild boars. Therefore, the combat of CSFV in the wild boar population is necessary for eliminating this source of infection for our domestic pig population.

For achieving a lasting health protection of our productive livestock—and last but not least for obtaining commercial advantages—the eradication of dangerous epizootics is aimed at in veterinary medicine and agriculture. Hence, the development of vaccines for animals, which can be used as prophylactics or, in the case of an epidemic, for preventing or minimizing a propagation of infectious diseases, has become a demand of vital importance for farmers and veterinarians.

The live vaccine against CSFV, which had been used for domestic pigs until vaccination with this substance was forbidden in 1992, helped to restrain the epidemic in the European countries and to prevent great economic damage in agriculture. The internationalization of the market, however, made it necessary to discontinue vaccinations against the classical epizootics so as to permit unrestricted trading in live animals and meat products within and out of the European Union. The background is that vaccinated animals and virus carriers cannot be differentiated serologically. Last year, a new generation of vaccines—so-called marker vaccines—for vaccinating domestic pigs against CSFV was submitted for approval. For this vaccination method, the recombinant viral proteins are to be produced in the baculo vector virus expression system (EP-A-O 924 298). Animals infected with a pestivirus develop antibodies (AB) against E^{rns}, E2 (both envelope proteins) and NS3 (a serine protease); only the antibodies directed against E2 have strong virus-neutralizing properties. After application of the marker vaccines, the pig only develops antibodies against the envelope protein contained in the vaccine and, by using a specific diagnostic test, it is then possible to differentiate between vaccinated and infected pigs. The production and primarily the application—each pig has to be vaccinated twice—require, however, much time and work and entail high costs. In addition, this vaccine cannot be administered orally, so that it cannot be used in the wild boar population

### BRIEF SUMMARY OF THE INVENTION

The present invention has been based on the technical problem of providing another method of producing marker vaccines, which does not show the disadvantages of the prior art. Another technical problem was to provide the means required for the above-mentioned process.

According to the present invention, the above-mentioned technical problems were solved by a process for producing a marker vaccine against a mammalian virus, which is characterized in that the immunogenic proteins of the vaccine are produced by a cereal plant or by parts thereof.

The further technical problem is solved by providing a transgenic cereal plant or parts thereof, containing at least one gene coding for an immunogenic protein of a mammalian virus.

### BRIEF DESCRIPTION OF THE DRAWINGS

FIG. 1 shows the vector pRS265.

FIG. 2 shows the vector pRS266.

FIG. 3 shows the vector pRS2600.

FIG. 4 shows the vector pRS268.

FIG. 5 shows the vector pAM300.

FIG. 6 shows the vector pRS269.

### DETAILED DESCRIPTION OF THE INVENTION

The production of heterologous products, especially heterologous polypeptides, in transgenic plant material has gained great importance during the last few years. In addition to the generation of transgenic plants, in which specific proteins are overexpressed or underexpressed with the aim of obtaining improved plant products by a modification of plant metabolism, it has well directed been attempted to construct transgenic plant material for producing heterologous products, which are normally not present in plants, e.g. bacterial toxins, human proteins or bioplastics.

The term “marker vaccine” specifies a vaccine leading to an immunization in the immunized organism, which differs from the immunization of the organism caused by the real pathogen. Normally, the marker vaccine produces a pattern of antibodies which is different from that produced by the real pathogen. It follows that, on the basis of the immune response obtained, it can be differentiated whether an organism has been confronted with a marker vaccine or with the natural pathogen.

A “cereal plant” in the sense of the present invention is a plant containing genetic information, normally due to a genetic change of the genotype, encoding at least one immunogenic protein—or at least immunogenic parts thereof—which is/are a constituent of the mammalian virus against which immunization in a mammalian organism is desired. The immunogenic protein is expressed by the cereal plant, if necessary, only after a specific induction of the promoter regulating the expression of the immunogenic protein.

Where appropriate, the immunogenic protein may also be expressed only in “parts” of the cereal plant This will normally be the case if the gene, which encodes the immunogenic protein, is under the regulation of a tissue-specific promoter. Consequently, the formulation “parts” of cereal plants specifies e.g. seeds, leaves and also tissue cultures of cereal plants.

In a preferred embodiment, a set of genes is expressed in the plant, said set of genes encoding part of the immunogenic proteins (or, more precisely, immunogenic epitopes) but not all the immunogenic proteins (epitopes) of the mammalian virus. This has the effect that the organisms, which have been immunized by means of the marker vaccine, produce an antibody pattern which is different from that produced by the organisms that have been infected with the natural pathogen. The marker vaccine can also be modified by adding an immunogenic component so that the added component leads to an additional immune response, which is not caused by the natural pathogen Such a component may be e.g. a protein which is not present in the natural pathogen, but which is added to the marker vaccine, thus causing in the treated organism also an antibody pattern that differs from the antibody pattern caused by the natural pathogen Such an additional protein, or immunogenic epitopes, thereof may be present e.g. as a fusion partner with the actual immunogenic protein.

In a further preferred embodiment, the cereal plant is barley, wheat, rye, triticale, rice or maize. Specially preferred cereal plants are those suitable for feeding animals, such as wheat, barley, maize.

In another preferred embodiment, the marker vaccine is suitable for immunizing against a virus from the family of pestiviruses, the immunization being especially an immunization against the classical swine fever virus, the border disease virus and the bovine viral diarrhea virus.

In another preferred embodiment, the immunogenic protein is selected from the gene product of the E2 gene or the E^{rns} gene of the classical swine fever virus. Derivatives of said gene products may be used as well, provided that they are still able to cause a sufficient immunization against said virus. The derivative may also consist of truncated forms of the full-length protein, provided that at least some of the immunogenic epitopes still exist The derivative may also be a fusion protein of the E2 or the E^{rns} gene product including a further protein, said further protein serving as the actual “marker”, as explained before. The derivative may also consist of mutants of the above-mentioned protein product, which have been optimized e.g. insofar as they cause an optimum immune response or insofar as the stability of the protein e.g. in the cereal plant has been improved.

In a further preferred embodiment, the codons of the gene or genes have been optimized in such a way that they are optimal for the cereal plant in which the marker vaccine is to be produced. This permits maximum expression of the desired protein in the cereal plant. A protein content which is as high as possible is also desirable with respect to the immunization properties of the marker vaccine produced from plants.

In another preferred embodiment, the expression of the immunogenic protein takes place only in parts of the plant, e.g. in the seeds; in this case, promoters are especially used which are exclusively active either during germination or during grain filling. Such expression systems are described e.g. in U.S. Pat. No. 5,693,506 as well as in WO 97/32986. This approach permits synthesizing the immunogenic protein in the plant or in the seed not already in the field but only during germination in a closed production chamber. This so-called malting technology deals with the germination of plant material under artificial conditions. Normally, this technology is used during the production of beer and comprises steps like steeping of the plant material and subsequent germination under controlled conditions. Under these “controlled” conditions, parameters such as humidity, temperature, salt concentration and agitation of the plant material are controlled by human intervention. Such processes are described, e.g. in “Die Bierbrauerei”, Vol. 1, “Die Technologie der Malzzubereitung”, Ferdinand Enke-Verlag Stuttgart (1999).

The present invention also relates to a transgenic cereal plant or parts thereof containing at least one gene encoding an immunogenic protein of a mammalian virus, the gene being a so-called transgene. Such “transgenic plant material” is a plant material which contains transgenic host cells, said transgenic host cells having at least one nucleic acid molecule stably integrated in their genome; this nucleic acid molecule is either heterologous with respect to the plant material, or it is normally not present in non-transformed cells, or it is homologous with respect to the plant material, but present in the transgenic cells in genomic surroundings which are different from those in wild-type cells.

In a preferred embodiment, the transgenic plant contains a set of genes encoding part of the immunogenic proteins of the mammalian virus. The idea underlying such a plant is that the plant does not express all the immunogenic epitopes of a pathogen, but only a subgroup, so that after an immunization with the subgroup of epitopes an antibody pattern is obtained which is different from the antibody pattern obtained when the organism has been infected with a pathogen that is intact. On the basis of the different immune responses between immunized organisms and infected organisms, the immunized organisms can be distinguished from the infected ones.

In another preferred embodiment, the plant is selected from barley, wheat, rye, triticale, rice and maize.

The plant preferably contains genes which code for proteins of a pestivirus.

In another preferred embodiment, the gene, which is expressed in the transgenic plants, encodes an immunogenic protein of a virus selected from the classical swine fever virus, the border disease virus and the bovine viral diarrhea virus. The genes of the surface proteins of said viruses are known and are available to the expert in the prior art.

Specially preferred genes for expression in the transgenic plant are the E2 gene and the E^{rns} gene of the classical swine fever virus; the gene can, e.g., by a purposeful mutagenesis, also be modified such that derivatives of the natural gene product are produced in the plant This may be advantageous e.g. for positively influencing the stability of the gene products and also the immunogenic properties thereof.

In order to obtain the highest possible expression yield of desired gene product in the plant, it will be of advantage to optimize the codons of the genes in such a way that they lead to maximum expression rates in the plant. Such codon optimizations are well known to the person skilled in the art for given hosts.

In a further preferred embodiment, the desired gene product is expressed especially in the grain of the plant. When a suitable promoter is used, which is activated e.g. under malting conditions, the desired gene product can thus be induced at an arbitrary time by induction of the respective promoter.

The present invention additionally relates to animal feed and food, respectively, which contain a cereal plant or parts thereof, as has been described hereinbefore. When administered e.g. orally, these compositions can then lead to a stimulation of the immune system, such as by inducing an immune response, against respective viruses and, desirably, provides protection upon challenge. The use of animal feed has, in particular, decisive advantages in comparison with the hitherto practiced techniques of vaccination by means of injection, since the feeding of animal feed can be carried out much more simply and is also easily possible e.g. for the immunization of non-domestic animals.

For an optimum yield of the expression product, the gene, which encodes a surface protein of a pathogen infecting animals, is subjected to “codon optimization”. In so doing, changes in the sequence encoding the gene are executed such that the codons of the wild type are replaced by the codons that are optimal for the host plant, without changing the amino acid sequence of the coded protein. In the examples, the optimum sequence for barley has been determined. The “chimeric gene” concerns a gene construct containing a DNA sequence which codes for a fusion protein, the gene construct containing a (i) N-terminal unit of a signal sequence and a (ii) protein component of a mature, heterologous protein, said protein component following directly the C-terminal part of the amino acid sequence of this signal sequence.

The immunogenic structural proteins used for expression originate from pestiviruses, the genus Pestivirus consisting essentially of the classical swine fever virus (CSFV), the border disease virus (BDV) and the bovine viral diarrhea virus (BVDV). The pestivirus genome consists of a positive stranded RNA of approx. 12.5 kb length containing a single open reading frame, which codes for a polyprotein of approximately 4,000 amino acids. The polyprotein is co- and post-translationally processed by cellular and viral proteases. The viral structural proteins, among them the envelope proteins E2, E^{rns} and E1, are located in the N-terminal part of the polyprotein. The native or synthetic gene can be modified with regard to an increased expression of the gene or the stability of the expressed proteins in the plant tissue. Methods that can be employed for changing the gene sequence are e.g. a directed mutagenesis, Exon shuffling and the like.

In a preferred construct, the chimeric gene product 5′ from the coding region comprises a promoter of a monocotyledonous plant Suitable promoters are the D-hordein promoter (SEQ ID NO: 3) and the amylase promoter (SEQ ID NO: 4) of barley, described e.g. also in Whittier R. F. et al. (1987) Nucleotide sequence analysis of alpha-amylase and thiol protease genes that are hormonally regulated in barley aleurone cells. Nucl. Acids Res. 15, 2515-2535 and Sorensen M. B. et al. (1996) Hordein promoter methylation and transcriptional activity in wild-type and mutant barley endosperm. Mol. Gen. Genef. 250, 750-60.

In addition, the chimeric gene contains, preferably between the promoter and the encoding sequence, the 5′-untranslated region (5′-UTR) and 3′ from the encoding sequence the untranslated region (3′-UTR) of a plant gene or of a bacterial gene. A preferred 3′-UTR is the nos terminator sequence. This sequence comprises the non-encoding sequence 5′ of the polyadenylation site, the polyadenylation site and the transcription termination sequence and is described e.g. in Jensen L. G. et al. (1998) Inheritance of a codon-optimized transgene expressing heat stable (1,3-1,4)-beta-glucanase in scutellum and aleurone of germinating barley. Hereditas 129: 215-225.

In the framework of the transformation of the plants, the chimeric gene of the present invention is introduced in a suitable expression vector for plants. The vector contains special elements of plasmids or viruses which permit DNA to be transported by the bacteria into the plants. Such expression vectors for plants are described e.g. in U.S. Pat. No. 5,693,506.

In addition to the chimeric genes described in this context, the vectors contain selection markers for use in plant cells, e.g. the nptII kanamycin resistance gene or the phosphinotricin acetyltransferase gene, as well as sequences allowing a selection and a growth in secondary hosts, such as yeast or bacteria.

In addition, the vectors used for this purpose contain regions which are homologous to an Agrobacterium tumefaciens vector, in the present case especially the T-DNA boundary sequences.

Transformation experiments with so-called double-cassette vectors permit the necessary “selection gene” to be separated from the desired transgene by outcrossing. This is especially important for proteins which, as in the present project, are to be applied orally. On these double cassette vectors both the selection gene, e.g. the bar gene, and the above-characterized chimeric gene are flanked by respective separate T-DNA boundary sequences so that the probability of an occurrence of independent recombinational events resulting in the plant genome is given.

The transformation of the plant material can preferably, but not exclusively, be executed according to the instructions given in U.S. Pat. No. 5,693,506 or WO 9,836,085: embryos are dissected from unripe seeds and, after 48 hours of incubation, they are co-cultivated with transformed Agrobacteria. This is followed by various washing steps and incubation first in the dark, later on in the light.

After respective selection steps, the selected plants are grown and cultivated. The expression rate of the foreign protein is determined first in unripe and later on in ripe grain.

Immunization is carried out preferably in the case of domestic pigs or wild boars against a viral infection, preferably against classical swine fever. This immunization comprises the transformation of plant material with an above-defined vector, the growing and cultivating of the transformed plant, the harvest of the desired plant material as well as the subsequent, preferably repeated feeding of the harvested material, e.g. barley grains or barley malt, to the animals; the material is either untreated or malted. Alternatively, the plant material protein encoded by the DNA of the vector can be purified and administered to the animals as fodder supplines.

Consequently, immunization by means of the processes described in the present connection comprises the following steps:

(1) constructing codon-optimized genes which encode immunogenic surface proteins of viral pathogens;

(2) transforming cereal plants with one or several, but not all, gene(s) for (an) immunogenic surface protein(s) of viral pathogens.

(3) characterizing, growing and cultivating the transgenic cereal plants;

(4) harvesting the grains;

(5a) feeding the grains to the animals as fodder or fodder supplies, or

(5b) malting the barley grains and feeding the malt to the animals as fodder or fodder supplies.

### Short Description of the Sequences

SEQ ID NO: 1 is the amino acid sequence of the E2 structural protein of the CSFV virus

SEQ ID NO: 2 is the nucleic acid sequence of the codon-optimized E2 gene of the CSFV virus

SEQ ID NO: 3 is the sequence of the D-hordein promoter of barley (grain filling)

SEQ ID NO: 4 is the sequence of the amylase promoter (germination)

### EXAMPLES

### Example 1

Construction of the Codon-Optimized E2 Gene of the CSFV Virus

The codons of the gene for the structural protein E2 of the CSFV virus are optimized such that the respective suitable GC-rich triplets are used for the individual amino acids. For this purpose, the gene is recombined from a total of 56 oligonucleotides (54 with a length of 40 nucleotides each and two with a length of 20 nucleotides). In the two oligonucleotides of the upper and lower strand which contain the stop codon, a Sac I restriction site for future cloning steps is inserted directly after the stop codon The oligonucleotides are combined by means of ligation and PCR.

The codon-optimized gene is placed by means of a “splice PCR” (cf. e.g. Horton et al., 1989, Gene 77: 61-68) behind a 3′ fragment of the hordein promoter with a hordein signal peptide, which contains an Nco I restriction site in the front part.

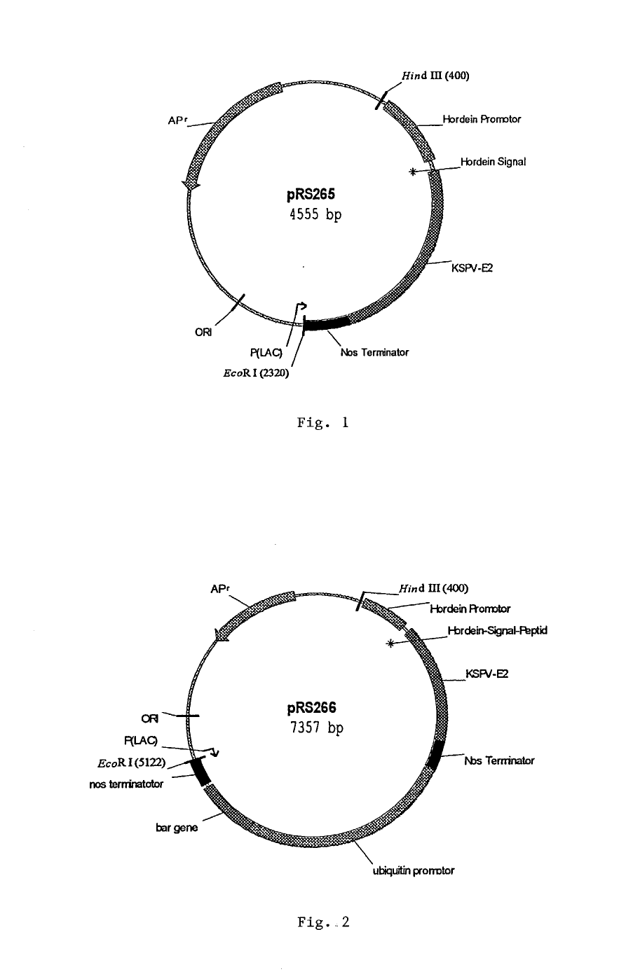

After cloning into a pUC18-vector, this fragment (consisting of the rear part of the hordein promoter, the hordein signal peptide and the complete E2 gene) is excised via Nco I and Sac I restriction sites and cloned into a vector containing a complete hordein promoter, a hordein signal peptide and an nos terminator. After the cloning, a construct (RS265; FIG. 1) is obtained, which consists of the hordein promoter, the hordein signal peptide, the E2 gene and the nos terminator in pUC18.

### Example 2

Construction of CSFV-Virus E2-Expressing Vectors as Single and Double Cassettes

Starting from the construct RS265, two vectors suitable for the transformation of an Agrobacterium of the AgII strain are now combined.

One construct comprises a so-called single cassette, the other a double cassette which differs from the single cassette insofar as the two chimeric genes (1) ubiquitin promoter, marker gene, nos terminator and (2) hordein promoter, hordein signal peptide, E2 gene, nos terminator are each surrounded by a left and a right T-DNA boundary region so as to permit a future independent integration in the plant genome. In the case of the single cassette, the whole unit consisting of ubiquitin promoter, marker gene, nos terminator and hordein promoter, hordein signal peptide, E2 gene, nos terminator is only surrounded by a respective common T-DNA boundary region.

Construction of the Single Cassette

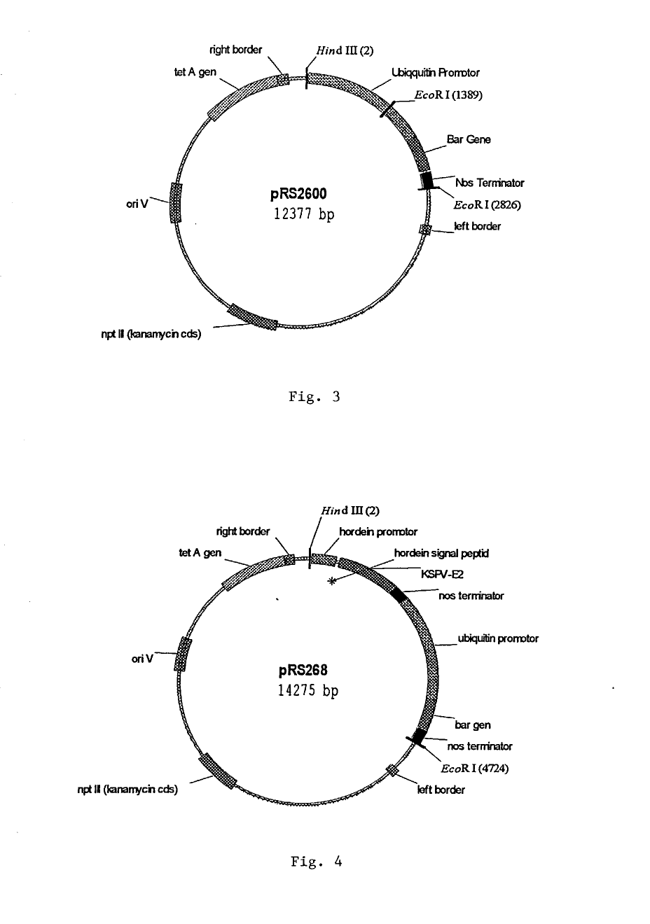

The above-described construct RS265 is first cleaved with Eco RI, whereupon the overlapping ends are filled by a T4 DNA polymerase treatment, and finally a Hin dIII cleavage is carried out. The purified fragment, on which the E2 gene is localized, is ligated in a pUC18 vector which contains a unit consisting of ubiquitin promoter, marker gene and nos terminator. This vector is split with Bam HI and Hin dIII prior to ligation, and also in this case the Bam HI restriction site is filled before the Hin dIII fission is carried out. After this cloning, the vector RS266 (FIG. 2) is obtained, the whole unit consisting of ubiquitin promoter, marker gene, nos terminator and hordein promoter, hordein signal peptide, E2 gene of the CSFV virus, nos terminator being excised from said vector by means of Eco RI/Hin dIII and inserted into an appropriately cut pRS2600 vector (FIG. 3). With this construct RS268 (FIG. 4) Agrobacteria of the strain AgII are transformed by means of electroporation.

Construction of the Double Cassette

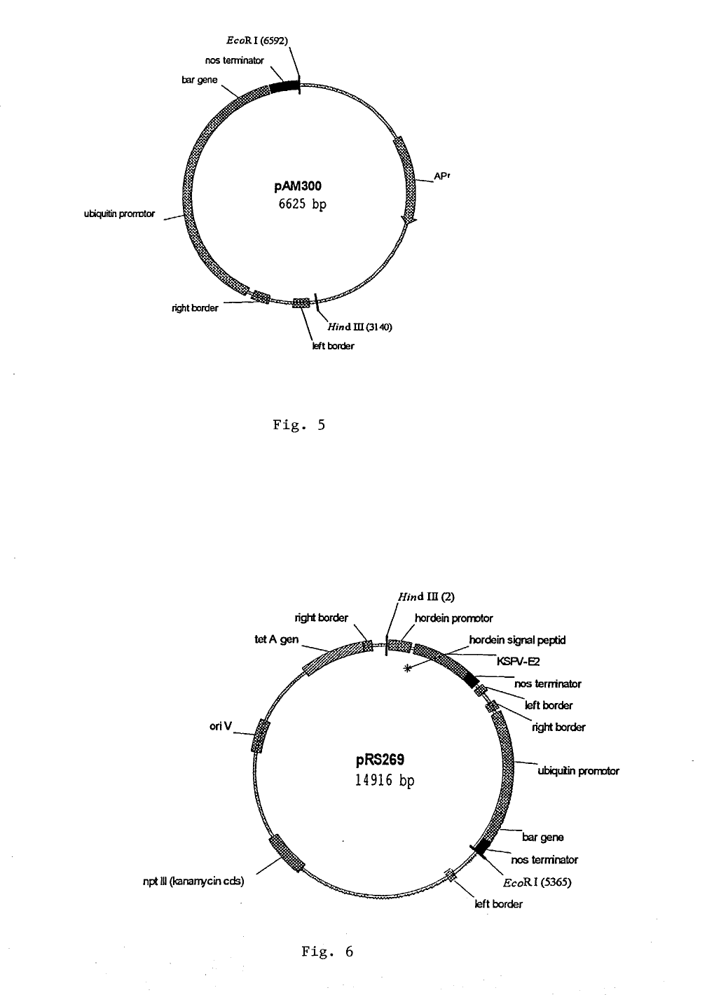

The above-described construct RS265 has excised therefrom the fragment with the E2 gene by means of Hin dIII and Eco RI, the Eco RI restriction site being again filled, as has been described above, before the Hin dIII cleavage is carried out. The vector pAM300 (FIG. 5) which is specially constructed for establishing double cassettes, already contains a left and a right T-DNA boundary region and a unit consisting of ubiquitin promoter, marker gene and nos terminator. This vector is cut with Not I/Hin dIII, the Not I restriction site being filled here. Subsequently, the Hin dIII/Eco RI fragment of the RS265 is cloned into this vector. Following this, a unit consisting of the ubiquitin promoter, marker gene, nos terminator and hordein promoter, hordein signal peptide, E2 gene, nos terminator, separated by a respective left and a respective right boundary sequence, is excised by means of Eco RI and Hin dIII and inserted into the suitably cut pRS2600. With this construct RS269 (FIG. 6) Agrobacteria of the AgII strain are transformed by means of electroporation.

### Example 3

Agrobacterium-Mediated Transformation of the Barley Line Golden Promise

Barley plants—preferably of the variant Golden promise—are cultivated e.g. in climatic chambers. After approx. 2-3 months, unripe barley seeds, whose embryos have a diameter of approx. 1.5 mm-2.5 mm, can be harvested.

The barley seeds are sterilized and, after a washing step, dissected from the grains and divided in the longitudinal direction. Under certain circumstances, it may here make sense to divide the embryos before they are dissected, by simply dividing the seed grain. The divided embryo is in this case dissected after the cutting. The embryos are incubated at 24° for 48 hours.

Agrobacteria of, e.g., the AgII strain, which have been transformed with the above-described transformation vectors, are grown in a selection medium at room temperature for 48 hours. After the incubation, the plant material is infected with the bacterial suspension. The incubation time of the Agrobacteria with the barley embryos at 24° C. in an aerated incubation chamber depends on the growth of the Agrobacteria; normally, it is, however, between 48 h and 72 h.

After the co-cultivation, the barley embryos are washed repeatedly until a turbidity of the medium caused by bacteria is no longer visible. After transplantation of the embryos to the selective plates, said plates are incubated in an aerated incubation chamber in the dark at 24° C. for two weeks. The selective plates comprise a growth hormone and a selection agent, e.g,. bialaphos. After two weeks, the embryos are transplanted to new plates and only sprouts and roots are roughly removed. The plates are again incubated in the dark at 24° C. for two weeks.

After two weeks, the embryos are transplanted again and, subsequently, grown under light which is especially suitable for plants. In this phase, the calli are intended to develop green areas. When the young plants are big enough, they are replanted into bowls where they can develop roots.

All publications, patents and patent applications cited herein are hereby incorporated by reference (including German patent application no. 101 45 969.6, filed Sep. 18, 2001, to which this application claims priority) to the same extent as if each individual document were individually and specifically indicated to be incorporated by reference and were set forth in its entirety herein.

While this invention has been described with an emphasis upon preferred embodiments, it will be obvious to those of ordinary skill in the art that the preferred embodiments may be varied. It is intended that the invention may be practiced otherwise than as specifically described herein. Accordingly, this invention includes all modifications encompassed within the spirit and scope of the appended claims.

What is claimed is:

1. A method of producing a marker vaccine against a mammalian virus, which method comprises producing the immunogenic protein(s) of the vaccine in a cereal plant or a part thereof.

2. A process according to claim 1, characterized in that a set of genes is expressed in said plant, said set of genes encoding part of the immunogenic proteins but not all immunogenic proteins of the mammalian virus.

3. A process according to claim 1, characterized in that the cereal plant is selected from barley, wheat, rye, triticale, rice and maize.

4. A process according to claim 1, characterized in that the mammalian virus originates from the family of pestiviruses.

5. A process according to claim 4, characterized in that the virus is selected from the classical swine fever virus, the border disease virus and the bovine viral diarrhea virus.

6. A process according to claim 5, characterized in that the immunogenic protein is selected from the gene product of the E2 gene and/or E^{rns} gene of the classical swine fever virus or of derivatives thereof.

7. A process according to claim 1, characterized in that the gene or the genes is/are codon-optimized for cereal plants.

8. A transgenic cereal plant or parts thereof comprising at least one gene which encodes an immunogenic protein of a mammalian virus.

9. A transgenic plant according to claim 8, characterized in that it comprises a set of genes which codes for part of the immunogenic proteins of the mammalian virus.

10. A transgenic plant according to claim 8, characterized in that it is selected from barley, wheat, rye, triticale, rice and maize.

11. A transgenic plant according to claim 8, characterized in that the mammalian virus is a pestivirus.

12. A transgenic plant according to claim 11, characterized in that the mammalian virus is selected from the classical swine fever virus, the border disease virus and the bovine viral diarrhea virus.

13. A transgenic plant according to claim 12, characterized in that the immunogenic protein is the gene product of the E2 gene and/or E^{rns} gene of the classical swine fever virus or of derivatives thereof.

14. A transgenic plant according to claim 8, characterized in that the gene or the genes is/are codon-optimized for cereal plants.

15. A transgenic plant according to claim 8, characterized in that the gene is expressed in the grain of the cereal plant.

16. Animal feed or food comprising a cereal plant or parts thereof according to claim 8.

17. A method of inducing an immune response to a mammalian virus in a mammal, which method comprises administering to the mammal an immune response-inducing amount of a marker vaccine produced in accordance with the method of claim 1, whereupon an immune response to the mammalian virus is induced in the mammal.

18. The method of claim 17, wherein the mammal is a pig.

19. The method of claim 17, wherein the marker vaccine is administered orally.
